(19) United States
(12) Patent Application Publication
(10) Pub. No.: US 2021/0353545 A1
(43) Pub. Date: Nov. 18, 2021

(54) NANOPARTICLE/HYDROGEL COMPLEXT FOR DRUG CARRIER

(71) Applicant: Tgel Bio Co., Ltd., Seoul (KR)

(72) Inventors: Keun Sang OH, Daejeon (KR); Han Weon CHO, Seoul (KR); Chang Soon HWANG, Incheon (KR); Sun Jong KIM, Seoul (KR); Hye Sook CHUNG, Seoul (KR)

(21) Appl. No.: 17/288,688

(22) PCT Filed: Oct. 29, 2018
(86) PCT No.: PCT/KR2018/012936

§ 371 Date: Apr. 26, 2021

(30) Foreign Application Priority Data
Oct. 26, 2018 (KR) 10-2018-0129322

(51) Int. Cl. A61K 9/16; A61K 9/19

(52) U.S. Cl. CPC A61K 9/1682; A61K 9/1617; A61K 9/1641; A61K 9/1652; A61K 9/19; A61K 9/1623

(57) ABSTRACT

A method for preparing a nanoparticle/hydrogel composite for a drug carrier according to one embodiment of the present invention comprises the steps of: forming stabilized lipid-based nanoparticles by irradiating an ultrasonic wave on a mixture of phosphatidylcholine and polysorbate 80 and then mixing a first polymer and freeze-drying same; and forming a lipid-based nanoparticle/hydrogel composite by mixing a second polymer and hyaluronic acid with the lipid-based nanoparticles and freeze-drying same.

### TECHNICAL FIELD

The present invention relates to a nanoparticle/hydrogel complex for a drug carrier, and more specifically, to a nanoparticle/hydrogel complex for a drug carrier that can delay the drug release rate and extend the duration of the drug, thereby maximizing the therapeutic efficacy of the drug when it is applied with a drug carrier in which the nanoparticle/hydrogel complex is mixed with the drug and introduced into the body.

### BACKGROUND ART

Hydrated gel, referred to as hydrogel, is a network structure in which water-soluble polymers form three-dimensional crosslinks by physical bonds (hydrogen bonds, Van der Waals forces, hydrophobic interactions, or crystals of polymers) or chemical bonds (covalent bonds). It refers to a substance that may not dissolve in an aquatic solution and may contain a considerable amount of water. This hydrogel has high biocompatibility due to its high moisture content and physicochemical similarity with the extracellular matrix. Due to these properties, hydrogel has received considerable attention in medical and pharmacological applications.

In particular, hydrogel is advantageous in that the drug can be easily mounted on the porous structure of the hydrogel, and the drug is slowly released depending on the diffusion coefficient of the drug. Therefore, when hydrogel is applied in the field of drug delivery, it is possible to continuously maintain a specific drug concentration in surrounding tissues over a long period of time.

Despite these advantages of hydrogels, there are some drawbacks that limit practical applications. First, the low tensile strength of the hydrogel limits its application in areas that must withstand the load, and as a result, it disappears in a short time through an early dissolution process at the target point. In addition, there is a problem caused by the high moisture content and porosity of the hydrogel, which makes it is difficult to carry hydrophobic drugs, which are released relatively quickly. In addition, most of the hydrogels are difficult to inject into the body using the injection method, and there are cases where a surgical operation is required to apply the hydrogel to the body. Due to these shortcomings, the practicality of the hydrogel is limited in clinical use.

The research addressing these issues is divided into two main directions. One is to improve the interaction between the drug and the hydrogel, and the other is to slow the diffusion of the drug from inside of the hydrogel.

Several methods have been studied for controlling the rate of drug release by modifying the surface of a hydrogel or the microstructure of the entire gel. A relatively dense hydrogel matrix was formed to provide excellent mechanical properties, controllable properties, and to improve drug loading efficiency compared to conventional hydrogels.

The drug delivery system depends on the polymer concentration of the delivery system maintained in the affected area. Thus, after administration in the body, it is easily dissolved (or broken down), resulting in a rapid drug release form due to instability. In particular, when water-soluble drugs, protein drugs, or antibodies are filled therein, rapid drug release was caused due to excellent solubility in an aqueous solution, making it difficult to implement the desired drug effect. As a way to overcome this problem, repeated administration of drugs is required, and the cost of treatment is expected to increase due to expensive protein drugs or antibodies. In order to address this issue, various nanoparticles loaded with drugs were mixed in a temperature-sensitive polymer. However, the polymer was not easily mixed with the nanoparticles, and the efficacy was not significantly improved.

Therefore, there is a need for a study to develop a method that has excellent biocompatibility and can slow the drug release time and speed.

### DISCLOSURE

### Technical Problem

An objective of the present invention is to provide a nanoparticle/hydrogel complex for a drug carrier that can be easily injected locally and in the body and a method for its preparation.

An objective of the present invention is to provide a nanoparticle/hydrogel complex for a drug carrier that can make a sustained release according to delayed absorption of the gel after introduction into the body by forming a temperature-sensitive hydrogel to a complex polymer network structure and a method for its preparation.

An objective of the present invention is to provide a nanoparticle/hydrogel complex for a drug carrier that has excellent sustained-release characteristics even by simple mixing with a drug and a method for its preparation.

An objective of the present invention is to provide a nanoparticle/hydrogel complex for a drug carrier that has excellent sustained-release characteristics by introducing nanoparticles capable of interacting with a drug to simply mix with a water-soluble drug and a method for its preparation.

### Technical Solution

The method for preparing a nanoparticle/hydrogel complex for a drug carrier of one embodiment of the present invention comprises the steps of: forming a first mixture by mixing phosphatidylcholine with a first solvent; forming a second mixture including a first nanoparticle by mixing polysorbate 80 with the first mixture; irradiating the second mixture with ultrasonic waves to pulverize the first nanoparticle to prepare a second nanoparticle; forming a third mixture by mixing a first polymer with the second mixture irradiated with the ultrasonic waves; lyophilizing the third mixture to obtain a third nanoparticle powder; forming a fourth mixture by mixing the third nanoparticle powder with a second solvent; forming a fifth mixture by mixing a second polymer with the fourth mixture; forming a sixth mixture by mixing sodium hyaluronate with the fifth mixture; and forming a nanoparticle/hydrogel complex by lyophilizing the sixth mixture.

The method of preparing a nanoparticle/hydrogel complex carrying a drug of another embodiment of the present invention comprises the step of: mixing the complex prepared by the preparation method described above with a drug containing water for injection.

The nanoparticle/hydrogel complex of an additional embodiment of the present invention comprises nanoparticles comprising phosphatidylcholine, polysorbate 80, and a first polymer; an irregular sodium hyaluronate polymer network located between the nanoparticles; and a matrix including a second polymer in which the nanoparticles and the polymer network are embedded.

The drug carrier of yet another embodiment of the present invention comprises the nanoparticle/hydrogel complex as described above and a drug dispersed in the complex.

### Advantageous Effects

According to an embodiment of the present invention, it is possible to provide a nanoparticle/hydrogel complex for a drug carrier that maintains a sol state outside the body and maintains a gel state in the body.

According to an embodiment of the present invention, it is possible to provide a nanoparticle/hydrogel complex for a drug carrier that can be locally injected due to its nano-size.

According to an embodiment of the present invention, it is possible to provide a nanoparticle/hydrogel complex for a drug carrier that is uniformly mixed when mixed with a solvent such as water for injection containing a desired drug.

According to an embodiment of the present invention, it is possible to provide a nanoparticle/hydrogel complex for a drug carrier that can reduce the rapid absorption and metabolism of the administered effective drug, increase the half-life of the drug, and exert effective therapeutic efficacy at the administered local site, when administered into the body with a drug.

According to one embodiment of the present invention, it is possible to provide a nanoparticle/hydrogel complex for a drug carrier that can be decomposed in the body or easily released outside the body because a lipid-based nanoparticle derived from a natural substance is applied thereto.

According to one embodiment of the present invention, it is possible to provide a nanoparticle/hydrogel complex for a drug carrier that can have excellent sustained-release characteristics even though it is simply mixed with a drug.

According to one embodiment of the present invention, it is possible to provide a nanoparticle/hydrogel complex for a drug carrier that has excellent sustained-release characteristics even though it is simply mixed a water-soluble drug, because it introduces the nanoparticles capable of interacting with drugs.

### DESCRIPTION OF DRAWINGS

FIG. 1 is a schematic diagram of a method of preparing a nanoparticle/hydrogel complex according to one embodiment of the present invention.

FIG. 2 is a schematic diagram of a drug delivery system when mixed with the drug in the case that a nanoparticle/hydrogel complex is used as a drug carrier according to one embodiment of the present invention.

FIG. 3 is a flow chart depicting a method of preparing a nanoparticle/hydrogel complex according to one embodiment of the present invention.

FIGS. 4A and 4B are graphs of average particle size and zeta potential of nanoparticles according to one embodiment of the present invention.

FIGS. 5A and 5B are graphs of average particle size and zeta potential of nanoparticles according to one embodiment of the present invention.

FIG. 6 is a graph of the average particle size and zeta potential of nanoparticles according to one embodiment of the present invention.

FIGS. 7A and 7B are images of a nanoparticle/hydrogel complex received in a container according to one embodiment of the present invention and a nanoparticle/hydrogel complex uniformly mixed in an aqueous solution.

FIG. 8 is a graph showing a change in gel formation temperature of a control group and a nanoparticle/hydrogel complex according to one embodiment of the present invention.

FIG. 9 is a graph showing a change in gel formation temperature according to the concentration of a nanoparticle/hydrogel complex according to one embodiment of the present invention.

FIG. 10 is an image depicting a method for indirectly checking physical properties through flowability while gradually changing to a sol state when various samples are formed into a gel at 37° C. and then the temperature is changed to room temperature.

FIG. 11 is a graph showing the amount of drug released versus time for various samples.

FIG. 12 is a graph showing the amount of drug released versus time for various samples.

### MODES OF THE INVENTION

The terms used in the present application are only used to describe specific manifestations and are not intended to limit the present invention. Singular expressions include plural expressions unless the context clearly indicates otherwise. In the present application, terms such as “include” or “have” are intended to designate that features, elements, etc. described in the specifications exist, but not to indicate that one or more other features or elements may not exist or be added.

In addition, terms such as “first” and “second” used herein are merely used to distinguish parts to be described later from each other and do not limit the parts to be described later.

Unless otherwise defined, all terms used herein including technical and scientific terms that have the same meanings commonly used and understood by practictioners of the discipline to which the present invention belongs. Terms, such as those defined in a commonly used dictionary, should be interpreted as having a contextually consistent meaning in relation to the related technology and should not be interpreted as an ideal or possessing excessively formal significance unless explicitly defined in the present application.

In the present application, the term “nano” may refer to a size in a nanometer (nm) unit. For example, it may reference a size of 1 nm to 1,000 nm but is not limited thereto. In addition, in the present specifications, the term “nanoparticle” may refer to a particle having an average particle diameter in a nanometer (nm) unit, and for example, it may refer to a particle having an average particle diameter of 1 nm to 1,000 nm but is not limited thereto.

Hereinafter, the present invention is described in more detail through the figures.

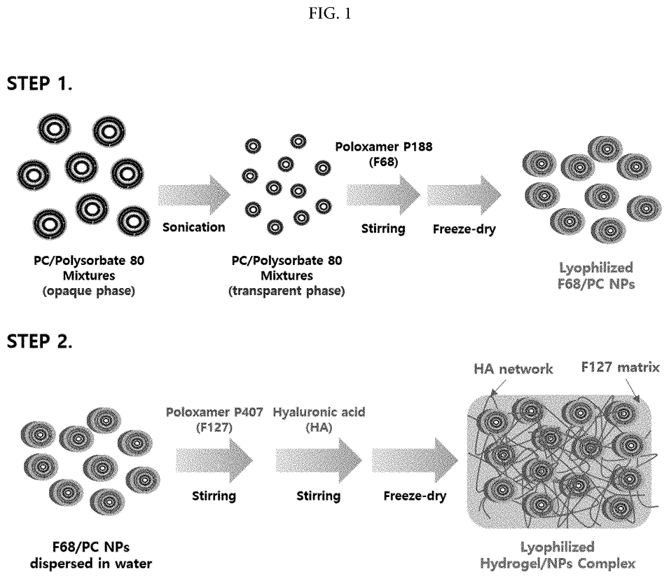

FIG. 1 is a schematic diagram of a method of preparing a nanoparticle/hydrogel complex according to one embodiment of the present invention.

As shown in FIG. 1, the method of preparing a nanoparticle/hydrogel complex for a drug carrier according to one embodiment of the present invention comprises steps of irradiating ultrasonic waves to a mixture of phosphatidylcholine and polysorbate 80, mixing the first polymer with the mixture, and freeze-drying to form lipid-based nanoparticles; and mixing the second polymer and sodium hyaluronate with lipid-based nanoparticles and freeze-drying to form a lipid-based nanoparticle/hydrogel complex.

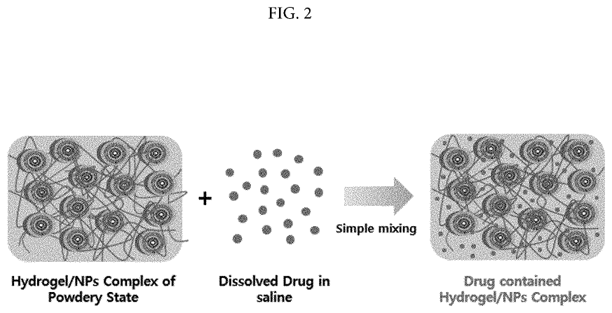

FIG. 2 is a schematic diagram of a drug delivery system when mixed with the drug in the case that a nanoparticle/hydrogel complex is used as a drug carrier according to one embodiment of the present invention.

As shown in FIG. 2, when the thus prepared nanoparticle/hydrogel complex for a drug carrier is used as a drug carrier to be mixed with a drug dissolved in an injectable agent, the drug can be uniformly dispersed in the substrate of the drug carrier.

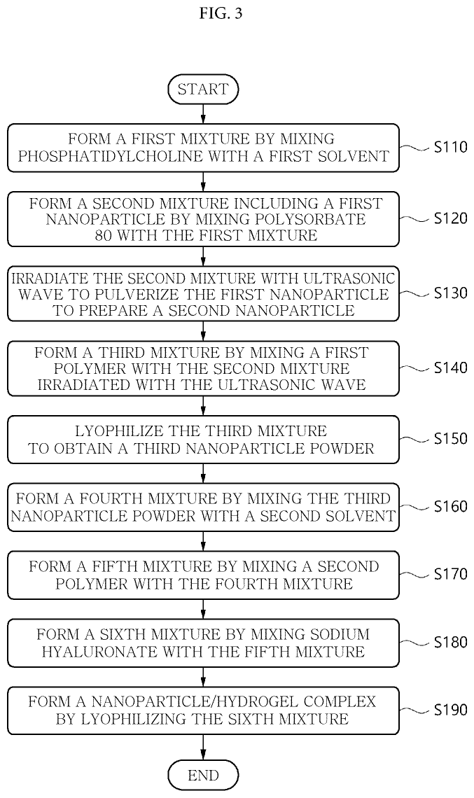

A method of preparing a drug carrier nanoparticle/hydrogel complex is described in more detail with reference to FIG. 3, which is a flow chart for a method of preparing a nanoparticle/hydrogel complex according to one embodiment of the present invention.

As shown in FIG. 3, first, the phosphatidylcholine is mixed with a first solvent to form a first mixture (S110).

The first solvent is not particularly limited, but any solvent suitable for dissolving phosphatidylcholine may be applied. For example, distilled water may be used as the first solvent.

Phosphatidylcholine is a phospholipid called lecithin, which is a combination of diglyceride and choline phosphate. This is a major constituent of biomembrane lipids, and it is a material that has excellent biocompatibility.

This phosphatidylcholine is mixed and dispersed in a first solvent to form the first mixture.

Then, polysorbate 80 is mixed with the first mixture to form the second mixture, which includes the first nanoparticle (S120).

Polysorbate 80 is an additive used to uniformly disperse liquids or solids that are not well mixed with each other in a liquid, and it may be used as an emulsifier and a stabilizer. This polysorbate 80 is added to the first mixture, sufficiently dissolved and uniformly dispersed, to form the second mixture.

In forming the second mixture, the mixing ratio (weight ratio) of the phosphatidylcholine and polysorbate 80 is preferably 1:0 to 2 (excess 0). While this mixing ratio will be described in more detail in the following experimental examples, the average size of the first nanoparticle can be controlled by the polysorbate 80, as intended by the present invention. In particular, the average size of the first nanoparticle may be controlled to 40 nm to 120 nm.

The second mixture thus formed is in an opaque state.

Then, the second mixture is irradiated with ultrasonic waves to pulverize the first nanoparticles to form the second nanoparticles (S130).

The method of irradiating with ultrasonic waves is not particularly limited, and a known ultrasonic irradiation method can be applied, and any method can be used unless the method deviates from the scope of the intended invention. Preferably, a probe-type ultrasonic irradiation method may be used.

Through irradiation with ultrasonic waves, the first nanoparticle is crushed to form the second nanoparticle, which is smaller in size. The third mixture, which is formed through this step of ultrasonic irradiation, is transparent, unlike the opaque second mixture.

Then, the first polymer is mixed with the second mixture that has been irradiated with ultrasonic waves to the third mixture (S140).

The first polymer is included to promote stabilization through modification of the surface of the second nanoparticle. In this case, the first polymer may be poloxamer 188. Poloxamers come with a 3-digit number. Multiplying the first two digits by 100 gives an approximate molecular weight of the polyoxypropylene core, and multiplying the last digit by 10 indicates the content of polyoxyethylene. Poloxamer 188 exhibits a molecular weight of polyoxypropylene of 1800 g/mol and polyoxyethylene content of 80%.

In forming the third mixture, the mixing ratio (weight ratio) of the second nanoparticles and poloxamer 188 is preferably 1:2 to 10.

It is preferable to stir the first polymer so that it is sufficiently mixed. The method of stirring is not particularly limited, and any method capable of achieving the intended result of the present invention may be applied.

The third mixture is lyophilized to obtain a third nanoparticle powder (S150).

The method of lyophilization is not particularly limited, and a known lyophilization method may be applied, preferably any method may be applied unless the method deviates from the scope of the intended invention.

Through this lyophilization, moisture contained in the third mixture may be evaporated, and finally, the lyophilized solid third nanoparticle may be obtained.

Then, the third nanoparticle powder is mixed with the second solvent to form the fourth mixture (S160).

The second solvent is not particularly limited, and any suitable solvent may be applied to dissolve the third nanoparticle. For example, distilled water may be used as the second solvent.

At this stage, it is preferable to ensure that the third nanoparticle is sufficiently stirred by stirring the fourth mixture until it becomes a transparent solution. The method of stirring is not particularly limited, and any method capable of achieving the intended result of the present invention may be applied.

Next, the second polymer is mixed with the fourth mixture to form the fifth mixture (S170).

Preferably, the second polymer may be poloxamer 407, which may be sufficiently stirred. The method of stirring is not particularly limited, and any method capable of achieving the intended result of the present invention may be applied.

Next, sodium hyaluronate is mixed with the fifth mixture to form the sixth mixture (S180).

In the case of a temperature-sensitive hydrogel composed of only poloxamer 407, the gel is well-formed when administered topically in the body, but most of it is absorbed into the body within one hour. However, when sodium hyaluronate is mixed and administered with the hydrogel, its rapid absorption in the body may be delayed due to the sodium hyaluronate network high molecular weight. When a drug (for example, a water-soluble or a non-water soluble drug) is administered together with sodium hyaluronate, the persistence of efficacy due to delayed absorption can be prolonged.

Meanwhile, it is possible to replace sodium hyaluronate with hyaluronic acid.

It is preferable that the sodium hyaluronate is sufficiently stirred. The method of stirring is not particularly limited, and any method capable of achieving the intended result of the present invention may be applied.

Next, the sixth mixture is lyophilized to form a nanoparticle/hydrogel complex (S190).

The method of lyophilization is not particularly limited, and any known lyophilization method may be applied, unless it deviates from the scope of the present invention.

By the method described above, it is possible to provide a nanoparticle/hydrogel complex for a drug carrier.

The method may further include the step of preparing a drug carrier with a drug by mixing a drug containing water for injection with the lyophilized nanoparticle/hydrogel complex.

The nanoparticle/hydrogel complex itself does not have any special efficacy in the body, but when a solution in a state that is simply mixed with a drug dissolved in water for injection is injected locally into the body, it changes into a gel form at body temperature. This allows the drug contained in the particle/hydrogel mixture complex to be slowly released.

The prepared nanoparticle/hydrogel complex does not have a therapeutic effect in the body, but may have a therapeutic effect through simple mixing with an injection solution that has been used in clinical practice.

The nanoparticle/hydrogel complex for a drug carrier according to one embodiment of the present invention includes a nanoparticle containing phosphatidylcholine, polysorbate 80, and the first polymer; an irregular sodium hyaluronate polymer network located between the nanoparticles; and a matrix including the second polymer in which the nanoparticles and the polymer network are embedded.

Descriptions of the same components as those described in the method of preparing the nanoparticle/hydrogel composite are excluded.

The complex comprises nanoparticles, a polymer network, and a matrix. Additional components may be included in this complex.

The nanoparticle is lipid-based, and the nanoparticle may interact with various drugs to be mixed later, so that the drugs are introduced into the body and thus slowly released.

Specifically, in the case of a hydrogel consisting only of poloxamer 407 or poloxamer 407/sodium hyaluronate, when carried through mixing with a drug, the polymeric substance and the drug interact with each other, so that the drug cannot be held for a certain period of time. Thus, the period of absorption and efficacy of the drug depends only on the absorption rate of the polymer gel in the body. Therefore, in the present invention, the nanoparticle/hydrogel complex for drug delivery is prepared to stabilize the lipid-based nanoparticles of natural ingredients capable of containing hydrophilic and hydrophobic drugs and to be mixed with hydrogel having a reversibly sol-gel behavior in response to temperature. Furthermore, through the interaction between the effective drug to be delivered and the stabilized lipid-based nanoparticles, it is possible to prolong the efficacy time by allowing the sustained drug release through a complex action rather than a system dependent only on the absorption rate of the gel in the body. In addition, phosphatidylcholine contained in nanoparticles is an amphoteric substance having both anionic and cationic properties, and it can be applied not only to nonionic salt-type drugs but also various ionic drugs (ex. doxorubicin, cisplatin, methotrexate, etc.) Thus, it has a strong interaction with lipid-based nanoparticles, which allows longer sustained release when administered into the body.

Lipid-based nanoparticles composed of phosphatidylcholine and polysorbate 80 have an average particle size of 40 nm to 100 nm in a hydrated state. The surface modification is performed using poloxamer 188 to stabilize the lipid-based nanoparticles without aggregation after freeze-drying. The average size of the hydrated particles by redispersing is formed from 250 nm to 300 nm. The average particle size is maintained even when nanoparticles stabilized with Poloxamer 188 are mixed with a temperature-sensitive hydrogel.

The polymer network contains sodium hyaluronate. The substance that determines temperature sensitivity is poloxamer 407. However, if it is composed of only poloxamer 407 or nanoparticles/poloxamer 407, the efficacy of the drug contained together cannot be sustained since it disappears after absorption in the body within 1 to 2 hours after administration. Meanwhile, when sodium hyaluronate is included, sodium hyaluronate exists in the form of a high molecular weight polymer network, so that the absorption rate in the body sustainably increases. Thus, the temperature-sensitive hydrogel containing sodium hyaluronate can also be sustainably absorbed to extend the absorption time of the drug contained, thereby extending the duration of the drug efficacy.

However, it is possible to replace sodium hyaluronate with hyaluronic acid.

The matrix consists of poloxamer 407. Through this matrix, it is possible to provide a complex in a sol state below body temperature or a gel state at body temperature.

When the drug is supported, the drug is uniformly dispersed in the nanoparticle/hydrogel complex. Through the interaction with the stabilized lipid-based nanoparticles, the drug may be carried or distributed in a form that coexists in other matrices.

Hereinafter, the present invention is described in more detail through experimental examples.

### Experimental Example 1. Analysis of Physicochemical Properties of Phosphatidylcholine/Polysorbate 80 Nanoparticles Dispersed in Aqueous Solution

First, 0.75 g of phosphatidylcholine was mixed and dispersed in 30 mL of tertiary distilled water at room temperature. Then, polysorbate 80 was mixed thereto. At this stage, the content of polysorbate 80 was varied to 0 g (T0), 0.1875 g (T25), 0.0.375 g (T50), 0.75 g (T100), 1.125 g (T150) and 1.5 g (T200). That is, compared to the phosphatidylcholine content, from 0 to 2 times polysorbate 80 was mixed. Then, the mixture was sufficiently stirred to obtain a homogeneous mixed solution. The mixed solution was in an opaque state.

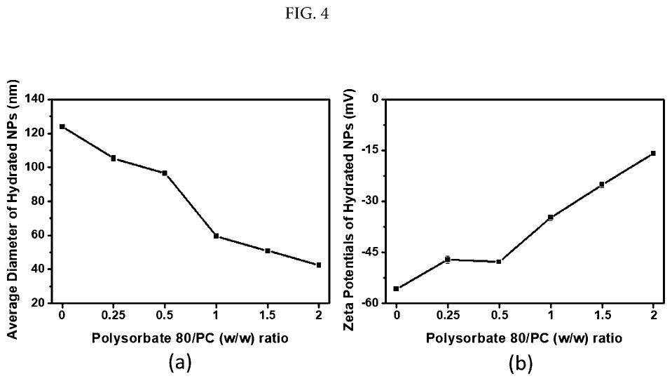

The average size and zeta potential of nanoparticles according to the ratio of phosphatidylcholine (PC) and polysorbate 80 were measured and shown in Table 1 below, and the graphs for the average particle size and zeta displacement are shown in FIGS. 4A and 4B.

TABLE 1

| Polysorbate | Hydrated NPs (D.W.) | |
|---|---|---|
| 80/PC (w/w) | Average Diameter | Zeta potential |
| ratio | (Average ± S.E. nm) | (Average ± S.E. mV) |
| 0 (T0) | 124 ± 1.27 | −55.75 ± 0.59 |
| 0.25 (T25) | 105.37 ± 1.72 | −47.19 ± 1.04 |
| 0.5 (T50) | 96.44 ± 0.21 | −47.8 ± 1.04 |
| 1 (T100) | 59.61 ± 0.31 | −34.78 ± 0.55 |
| 1.5 (T150) | 50.87 ± 0.27 | −25.07 ± 0.81 |
| 2 (T200) | 42.48 ± 0.36 | −15.85 ± 0.47 |

As shown in Tables 1 and 4, as the content of polysorbate 80 increases, the size of the particles decrease, and the zeta potential (surface charge) also decreases. Through this evidence, it can be confirmed that the size and surface charge of the nanoparticles can be controlled according to adjustment of the content of polysorbate 80.

### Experimental Example 2. Analysis of Physicochemical Properties of Phosphatidylcholine/Polysorbate 80/Poloxamer 188 Lipid-Based Nanoparticles Dispersed in Aqueous Solution

In order to analyze the physicochemical properties of the phosphatidylcholine/polysorbate 80/poloxamer 188 lipid-based nanoparticles dispersed in an aqueous solution, phosphatidylcholine/polysorbate 80 nanoparticles prepared in Experimental Example 1 were used. 20% of poloxamer 188 was mixed with the nanoparticles. At this stage, the ratio of the poloxamer 188 and the lipid-based nanoparticles was added while controlling at 10:1, 10:3, and 10:5. It was then stirred and freeze-dried to obtain phosphatidylcholine/polysorbate 80/poloxamer 188 nanoparticles.

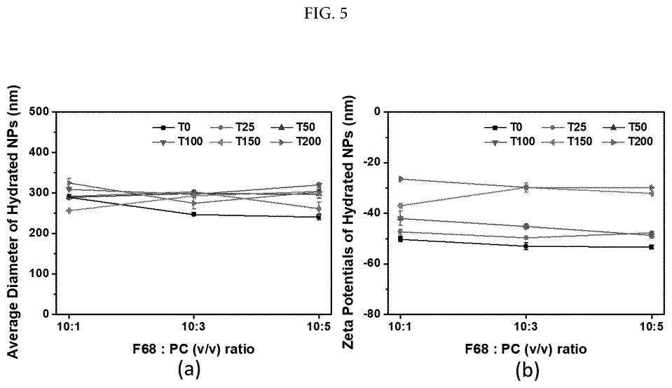

After re-dispersing the phosphatidylcholine (PC)/polysorbate 80/poloxamer 188 (F68) nanoparticles in distilled water, the average size and zeta potential were measured and shown in Table 2. Graphs of the average particle size and zeta displacement of the nanoparticles are shown in FIGS. 5A and 5B.

TABLE 2

| Polysorbate | F68:Polysorbate | Hydrated NPs (D.W.) | |
|---|---|---|---|
| 80/PC | 80/PC | Average Diameter | Zeta potential |
| (Weight | (Weight | (Average ± | (Average ± |
| ratio) | ratio) | S.E. nm) | S.E. mV) |
| 0 (T0) | 10:1 | 289.80 ± 1.97 | −50.2 ± 1.11 |
| | 10:3 | 246.97 ± 3.90 | −53.1 ± 1.38 |
| | 10:5 | 240.70 ± 7.80 | −53.37 ± 0.79 |
| 0.25 (T25) | 10:1 | 292.10 ± 4.76 | −47.47 ± 0.99 |
| | 10:3 | 302.63 ± 3.53 | −49.7 ± 0.65 |
| | 10:5 | 260.73 ± 16.69 | −47.67 ± 0.20 |
| 0.5 (T50) | 10:1 | 289.80 ± 4.97 | −42.07 ± 2.86 |
| | 10:3 | 299.53 ± 5.86 | −45.23 ± 0.95 |
| | 10:5 | 297.07 ± 10.26 | −48.70 ± 1.19 |
| 1 (T100) | 10:1 | 309.23 ± 3.84 | −42.07 ± 2.86 |
| | 10:3 | 296.17 ± 6.93 | −45.23 ± 0.95 |
| | 10:5 | 319.67 ± 4.95 | −48.7 ± 1.19 |
| 1.5 (T150) | 10:1 | 255.97 ± 2.72 | −37.1 ± 0.75 |
| | 10:3 | 293.17 ± 0.90 | −29.83 ± 1.85 |
| | 10:5 | 304.40 ± 9.44 | −32.23 ± 0.22 |
| 2 (T200) | 10:1 | 324.63 ± 10.96 | −26.53 ± 0.67 |
| | 10:3 | 274.57 ± 14.38 | −29.87 ± 0.68 |
| | 10:5 | 302.60 ± 9.70 | −29.93 ± 0.33 |

As shown in Table 2 and FIGS. 5A and 5B, it can be confirmed that various lipid-based nanoparticles were stabilized by poloxamer 188 to be re-dispersed well without aggregation of the particles.

### Experimental Example 3. Analysis of Stability of Phosphatidylcholine/Polysorbate 80/Poloxamer 188 Nanoparticles in Water for Injection (0.9% NaCl)

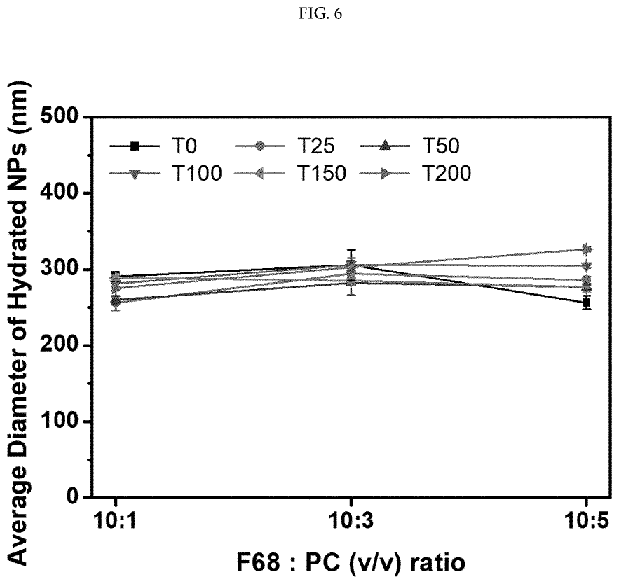

In order to confirm whether the phosphatidylcholine/polysorbate 80/poloxamer 188 nanoparticles remain stable in the water for injection, mg of the nanoparticles prepared in Experimental Example 2 were added and dispersed in the injection water containing 10 mL 0.9% NaCl. Particle size and surface charge were analyzed together using a particle size analyzer, and the measurement results are shown in Table 3 and FIG. 6.

TABLE 3

| | F68:Polysorbate | |
|---|---|---|
| Polysorbate | 80/PC | Hydrated NPs (0.9% NaCl) |
| 80/PC | (Weight | Average Diameter |
| (Weight ratio) | ratio) | (Average ± S.E. nm) |
| 0 (T0) | 10:1 | 290.53 ± 6.57 |
| | 10:3 | 305.37 ± 19.84 |
| | 10:5 | 256.43 ± 8.69 |
| 0.25 (T25) | 10:1 | 255.93 ± 9.39 |
| | 10:3 | 294.10 ± 4.84 |
| | 10:5 | 286.20 ± 4.75 |
| 0.5 (T50) | 10:1 | 259.97 ± 4.66 |
| | 10:3 | 282.43 ± 16.07 |
| | 10:5 | 276.47 ± 3.35 |
| 1 (T100) | 10:1 | 281.5 ± 3.97 |
| | 10:3 | 306.03 ± 8.97 |
| | 10:5 | 304.97 ± 3.35 |
| 1.5 (T150) | 10:1 | 288.97 ± 2.72 |
| | 10:3 | 285.2 ± 3.18 |
| | 10:5 | 277.1 ± 7.28 |
| 2 (T200) | 10:1 | 275.07 ± 2.61 |
| | 10:3 | 302.47 ± 6.85 |
| | 10:5 | 326.23 ± 2.77 |

As shown in Table 3 and FIG. 6, the phosphatidylcholine/polysorbate 80/poloxamer 188 nanoparticles were well redispersed without agglomeration of particles similar to the result of particle size change dispersed in tertiary distilled water.

### Experimental Example 4. Mixing of Nanoparticle/Hydrogel Complex with Water for Injection

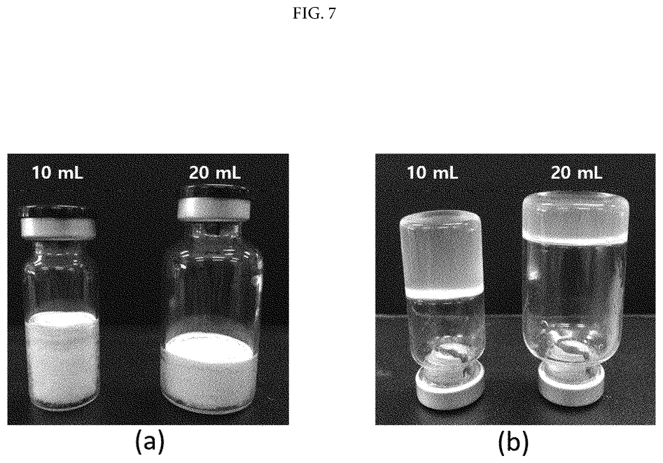

20 g of the nanoparticles prepared in Experimental Example 2 were added to 10 mL of distilled water. The mixture was stirred and sufficiently dispersed to be a transparent solution. Then 2 g of poloxamer 407 (F127) and 0.05 g of sodium hyaluronate (SH) were sequentially added and sufficiently mixed to prepare a uniform and transparent solution. Thereafter, this mixed solution was lyophilized to obtain a solid nanoparticle/hydrogel complex. The image of the nanoparticle/hydrogel complex in which the nanoparticle/hydrogel complex received in the container and the effective drug were uniformly mixed is shown in FIG. 7.

FIG. 7 shows that the nanoparticle/hydrogel complex was uniformly dispersed in the water for injection.

### Experimental Example 5. Analysis of Temperature Sensitivity of Nanoparticle/Hydrogel Complex

Additional experiments were conducted to confirm whether the nanoparticle/hydrogel complex, according to one embodiment of the present invention, can be reversibly converted to sol-gel by external temperature when it is dissolved at a certain concentration or higher in an aqueous solution such as distilled water or water for injection.

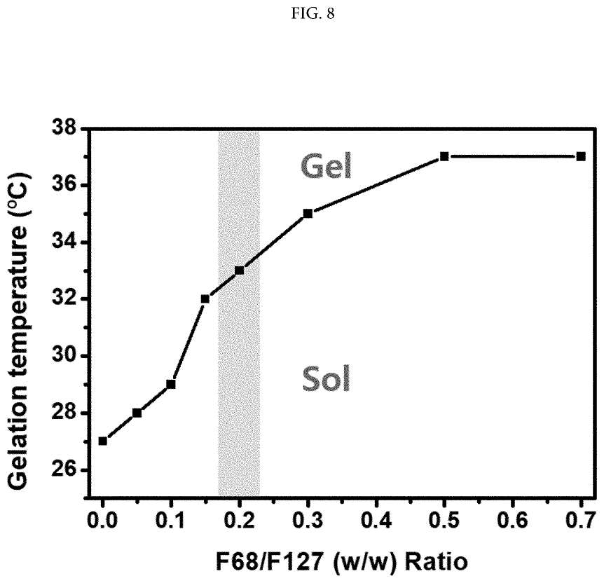

First, as a control group, only poloxamer 188 (F68), the main polymer used for stabilization of lipid nanoparticles, was mixed with a hydrogel containing poloxamer 407 (F127), and then the change in gel formation temperature was measured. A standard for the amount of nanoparticles to be added to the hydrogel was set based on the data. The change in gel formation temperature of the control group is shown in Table 4 and FIG. 8.

TABLE 4

| | | | Gel formation |
|---|---|---|---|
| Inactive ingredients | | | (gelation) |
| F127 | SH | F68 | Temperature (° C.) |
| 2 g | 0.05 g | 0 g | 27 |
| | | 0.05 g | 28 |
| | | 0.1 g | 30 |
| | | 0.15 g | 32 |
| | | 0.2 g | 33 |
| | | 0.3 g | 35 |
| | | 0.5 g | 37 |
| | | 0.7 g | 37 |

As shown in Table 4 and FIG. 8, the sample without poloxamer 188 (F68) turned into a gel at 27° C. On the other hand, when poloxamer 188 was added, the gel formation temperature gradually increased as the amount of poloxamer 188 was increased.

An additional experiment was conducted in order to apply the hydrogel in clinical practice. An appropriate mixing ratio of poloxamer 407 and poloxamer 188 that can be easily converted into a gel in response to body temperature after being in a sol state at room temperature and is easy to handle was set in which the amount of nanoparticles added was fixed at 10% compared to poloxamer 407.

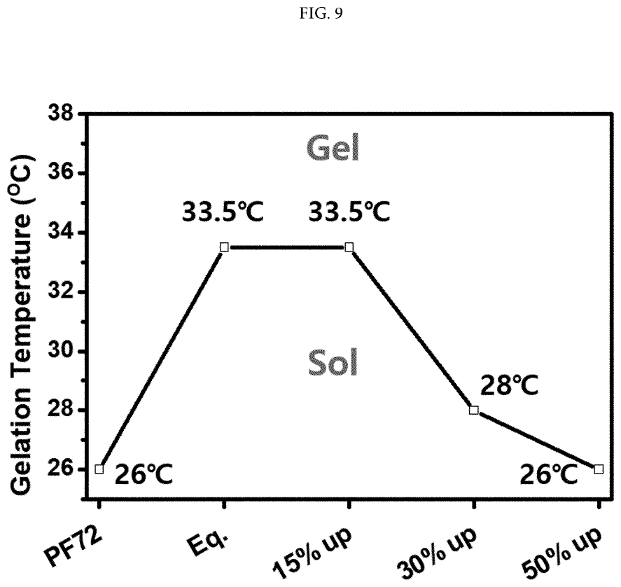

Based on the results shown in FIG. 8, a nanoparticle/hydrogel mixed complex was prepared. Specifically, the amount of nanoparticles compared to poloxamer 407 was fixed at 10%, and the gel formation temperature was measured while increasing the amount of poloxamer 188 (increasing concentration), which is shown in Table 5 and FIG. 9.

TABLE 5

| | | | | Gel formation |
|---|---|---|---|---|
| | | | | (gelation) |
| | Inactive ingredients | | | Temperature |
| Samples | F127 | SH | F68/PC NPs | (° C.) |
| PF72 | 2 g | 0.05 g | 0 g | 26 |
| Eq. | 2 g | 0.05 g | 0.2 g | 33.5 |
| 15% up | 2.3 g | 0.0575 g | 0.23 g | 33.5 |
| 30% up | 2.6 g | 0.065 g | 0.26 g | 28 |
| 50% up | 3 g | 0.075 g | 0.3 g | 26 |

As shown in Table 5 and FIG. 9, similar to the result of the control group, the complex (Eq.) in which the nanoparticles were mixed with the hydrogel had an increased gel formation temperature compared to the pure hydrogel (PF72). It can be confirmed that as the amount of the nanoparticle/hydrogel complex (Eq.) was gradually increased from 15% to 50%, the gel formation temperature may be lowered.

A substance having temperature-sensitive properties needs to change from a solution state to a gel state quickly and stably at body temperature. As shown in FIG. 9, these basic conditions can be satisfied as the total amount of the nanoparticles/hydrogel is increased.

### Experimental Example 6. Confirmation of Change in Physical Properties of Nanoparticle/Hydrogel Complex According to Temperature Change

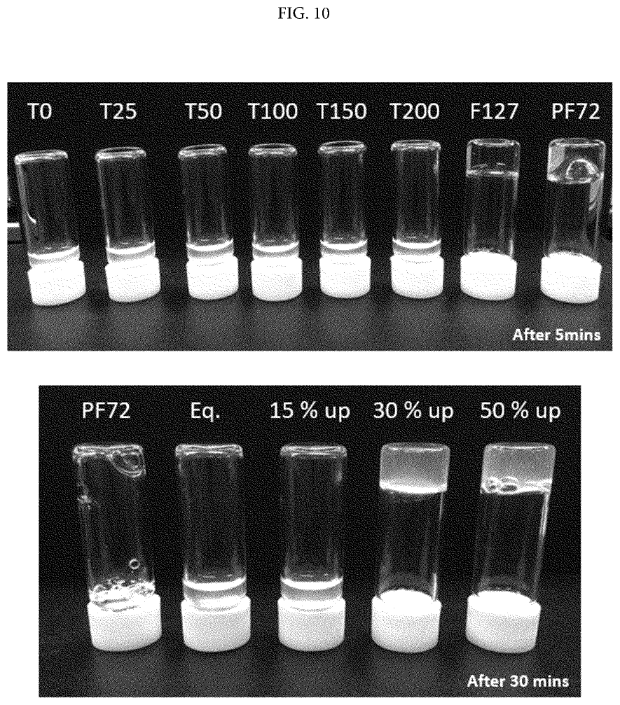

After being formed into a gel at body temperature, when it is easily changed into a sol state according to a change in external temperature, the physical properties of the gel are lowered. In order to confirm these physical properties of the complex according to one embodiment of the present invention, various samples were formed into a gel at 37° C. While the temperature was changed to room temperature, it gradually changed to a sol state, and its physical properties were indirectly confirmed through their flowability, which is shown in FIG. 10.

This change, as shown in FIG. 10, could be confirmed with the naked eye. Specifically, it can be confirmed that according to the amount of polysorbate 80 when forming nanoparticles, the prepared nanoparticle/hydrogel complex reacted sensitively to temperature changes to be changed into a sol state, compared to F127 and pure hydrogel (PF72) without nanoparticles. Meanwhile, it can be confirmed that the nanoparticle/hydrogel complex has good physical properties because the samples with 30% and 50% were among the samples in which the amount of the nanoparticle/hydrogel complex was increased from 15% to 50%, maintained the gel type despite the passage of time, compared to pure hydrogel (PF72).

### Experimental Example 7. Confirmation of Drug Release Behavior from Nanoparticle/Hydrogel Complex

Nacain injection solution (tradename) (active ingredient: ropivacaine, Naca Inj.) containing water for injection, which is widely used for local anesthesia of patients in clinical practice as a model drug, was simply mixed with the nanoparticle/hydrogel complex to carry the drug. Then, the drug release behavior was confirmed in vitro.

In order to evaluate the drug release behavior, the nanoparticle/hydrogel complex was selected as a sample in which a gel was formed at body temperature (37° C.) after being in a sol state at room temperature. As a control group, only Nacain injection in which pure ropivacaine was dissolved in water for injection, a group consisting of ropivacaine and poloxamer 407 (F127), a temperature-sensitive polymer, and a group consisting of ropivacaine, F127 and SH (the group does not contain nanoparticles). There was a total of 10 experimental groups, with 6 groups (T0, T25, T50, T100, T150 and T200) prepared while controlling the content of polysorbate 80 and 4 groups (Equ., 15% up, 30% up and 50% up) according to the increase in the amount of the nanoparticle/hydrogel complex. These samples were evaluated for comparison.

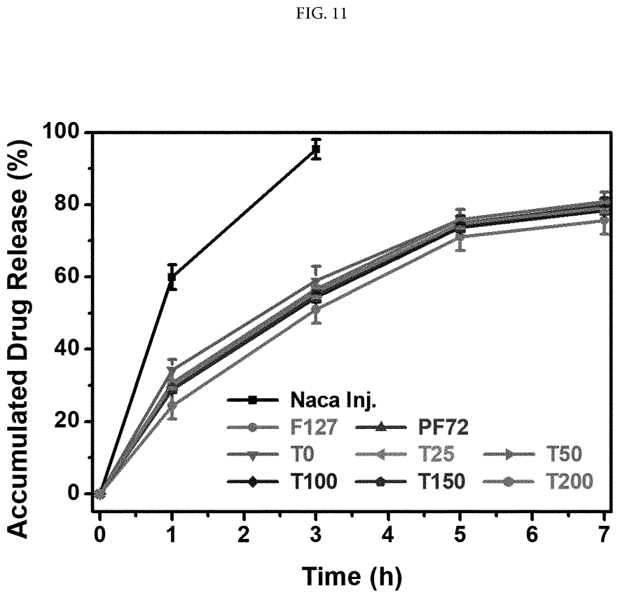

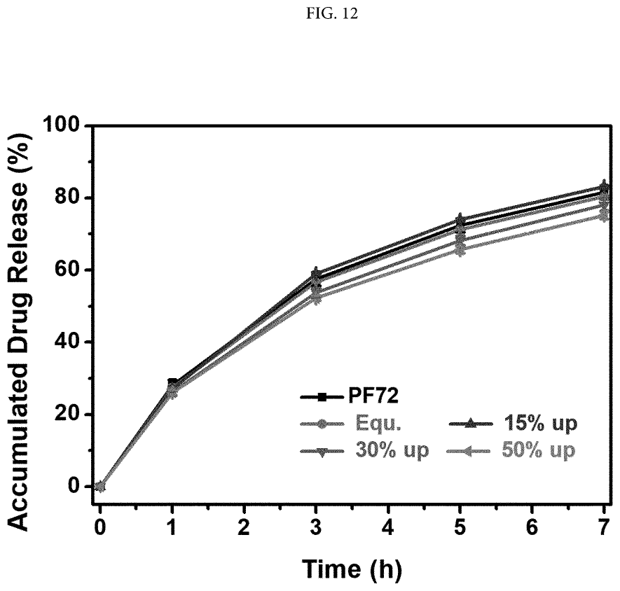

All the samples were kept refrigerated using crushed ice to maintain fluidity at room temperature until they were used in the experiment. A certain amount of each sample was injected into a semi-permeable dialysis bag and contained in a 50 mL centrifuge tube. After sealing, the entire samples were kept in an oven maintained at 37° C. for 30 minutes to form a gel. Then 30 mL of the buffer solution maintained at 37° C. was placed in a centrifugal tube so that the samples were immersed. The samples were then placed in an agitating thermostat (37° C., 50 rpm), and drug release was performed at predetermined times. The resulting data is shown in FIGS. 11 and 12.

As shown in FIG. 11, among the control groups, the drug group dissolved in water for injection was released most rapidly, and the other groups showed similar release behavior. It can be confirmed that the control group consisting only of poloxamer 407 (F1270) had slower release than that of the other groups. This is because it is a complex phenomenon in which the sample consisting only of poloxamer 407 maintained the high stability of gel formation at 37° C. with a lower gel formation temperature compared to other samples.

In addition, as shown in FIG. 11, as the amount of the nanoparticle/hydrogel complex was increased, the sustained-release drug was gradually released as compared to the PF72 control. These results are determined to occur because the gel formation temperature and gel retention stability are high according to the increase in the amount of the nanoparticle/hydrogel complex in the above-mentioned physicochemical properties (as shown in FIG. 10). Therefore, the results indicate that the drug release rate can be controlled.

Although described above with reference to preferred manifestations of the present invention, it should be understood that those skilled in the discipline will variously modify and change the present invention without departing from the spirit and scope of the present invention described in the following claims.

1. A method of preparing a nanoparticle/hydrogel complex for a drug carrier, the method comprising steps of:
forming a first mixture by mixing phosphatidylcholine with a first solvent;
forming a second mixture including a first nanoparticle by mixing polysorbate 80 with the first mixture;
irradiating the second mixture with ultrasonic wave to pulverize the first nanoparticle to prepare a second nanoparticle;
forming a third mixture by mixing a first polymer with the second mixture irradiated with the ultrasonic wave;
lyophilizing the third mixture to obtain a third nanoparticle powder;
forming a fourth mixture by mixing the third nanoparticle powder with a second solvent;
forming a fifth mixture by mixing a second polymer with the fourth mixture;
forming a sixth mixture by mixing sodium hyaluronate or hyaluronic acid with the fifth mixture; and
forming a nanoparticle/hydrogel complex by lyophilizing the sixth mixture.

2. The method of claim 1, wherein the first polymer is poloxamer 188.

3. The method of claim 1, wherein the second polymer is poloxamer 407.

4. The method of claim 1, wherein the second mixture is opaque.

5. The method of claim 1, wherein the third mixture is transparent.

6. The method of claim 1, wherein in step of forming the second mixture, the mixing ratio of the phosphatidylcholine and polysorbate 80 is 1:0 to 2 (excess 0).

7. The method of claim 1, wherein in step of forming the second mixture, the average size of the first nanoparticle is 40 nm to 120 nm.

8. The method of claim 1, wherein in step of forming the third mixture, the mixing ratio (weight ratio) of the second nanoparticles and the poloxamer 188 is 1:2 to 10.

9. (canceled)

10. (canceled)

11. A nanoparticle/hydrogel complex for a drug carrier, the complex comprising:
nanoparticles comprising phosphatidylcholine, polysorbate 80, and a first polymer;
an irregular sodium hyaluronate or hyaluronic acid polymer network located between the nanoparticles; and
a matrix including a second polymer in which the nanoparticles and the polymer network are embedded.

12. The complex of claim 11, wherein the nanoparticle/hydrogel complex is a sol state below body temperature, but a gel state at body temperature.

13. The complex of claim 11, wherein the first polymer is poloxamer 188.

14. The complex of claim 11, wherein the nanoparticles are lipid-based nanoparticles.

15. The complex of claim 12, wherein the matrix is immobilized in a gel state.

16. The complex of claim 11, wherein the second polymer is a poloxamer 407.

17. The complex of claim 11, wherein the content ratio of the second polymer and the first polymer is 1:0.1 to 0.7.

18. (canceled)

19. A drug carrier comprising the nanoparticle/hydrogel complex of claim 11, and a drug dispersed in the complex.
